(19) United States
(12) Patent Application Publication
(10) Pub. No.: US 2003/0083225 A1
(43) Pub. Date: May 1, 2003

(54) Organolipic substance

(76) Inventors: Claus-Herrmann Kappey, Holzminden (DE); Bernd Holscher, Halle (DE); Manfred Meier, Furstenberg (DE); Norbert Andreas Braun, Holzminden (DE); Berthold Weber, Holzminden (DE); Wilhelm Pickenhagen, Hoxter (DE)

Correspondence Address: PENDORF & CUTLIFF, 3940 Venetian Way, Tampa, FL, 33634, US

(21) Appl. No.: 10/243,597

(22) Filed: Sep. 12, 2002

(30) Foreign Application Priority Data
Sep. 12, 2001 (DE) 101 44 816.3

(51) Int. Cl. A61K 7/46

(52) U.S. Cl. 512/14

(57) ABSTRACT

Described is the compound of the Formula

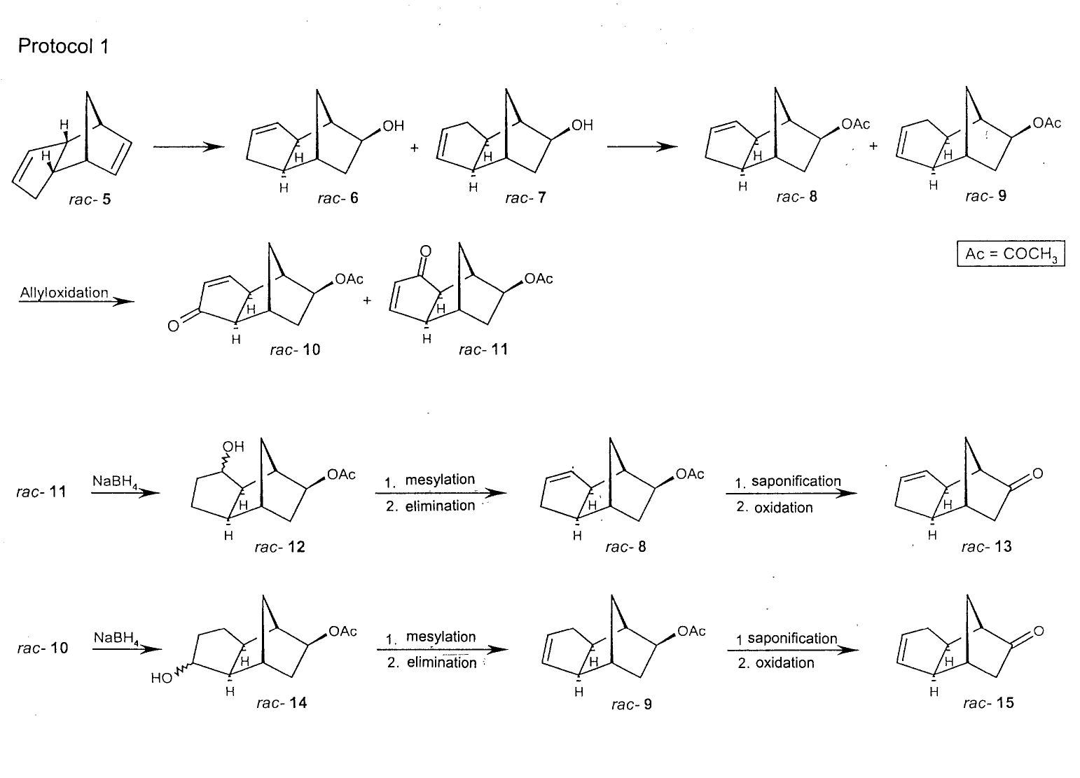

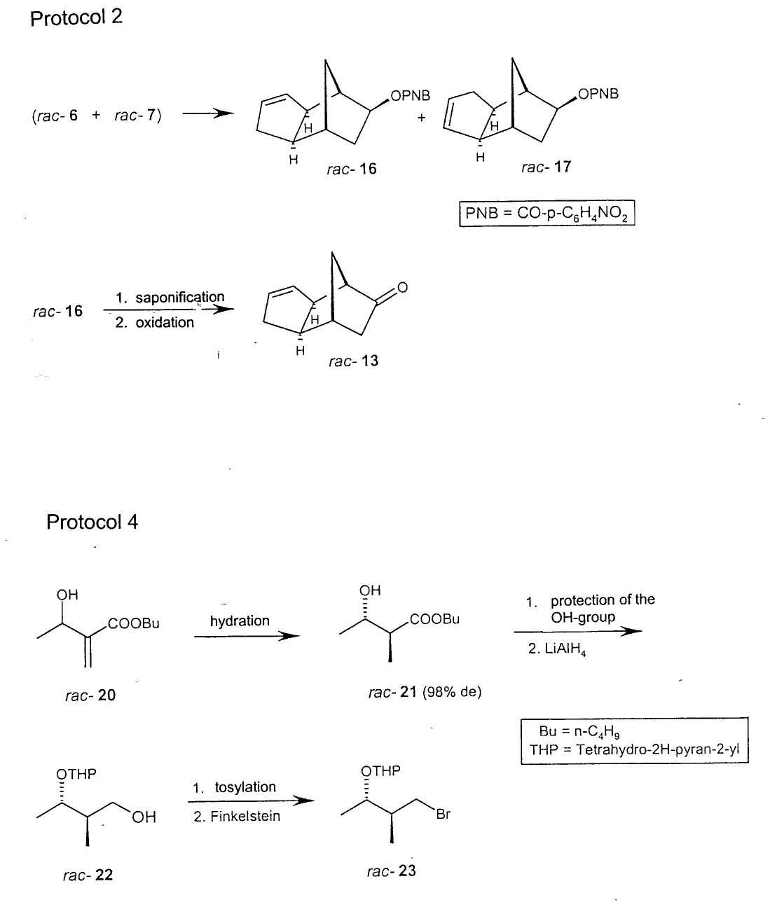

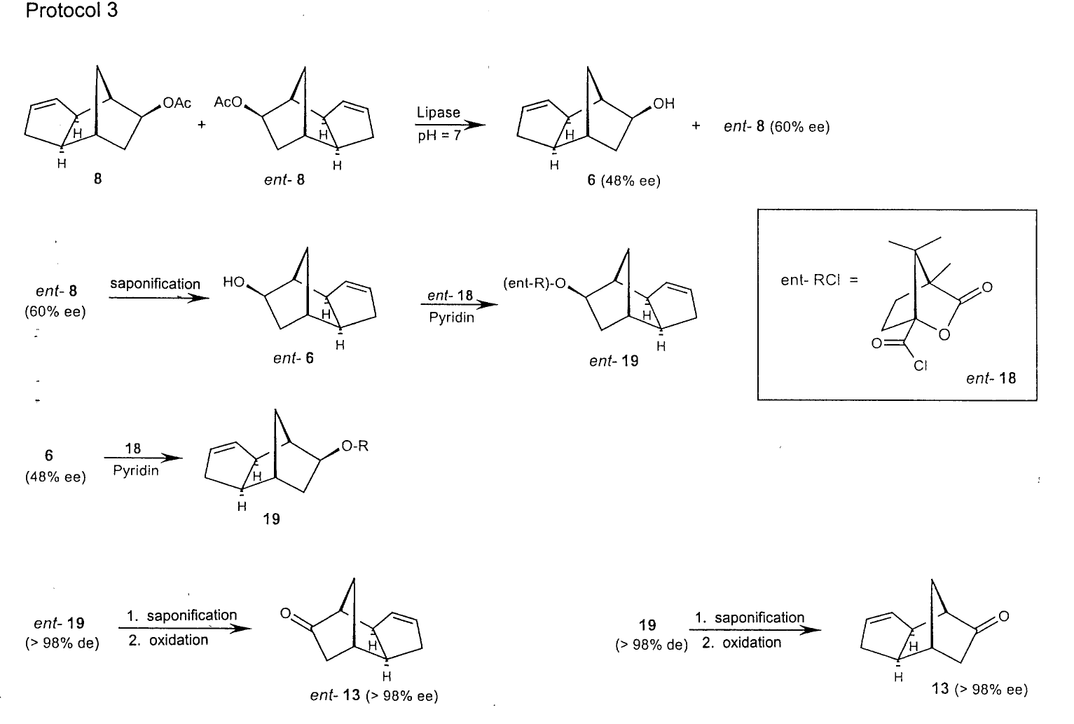

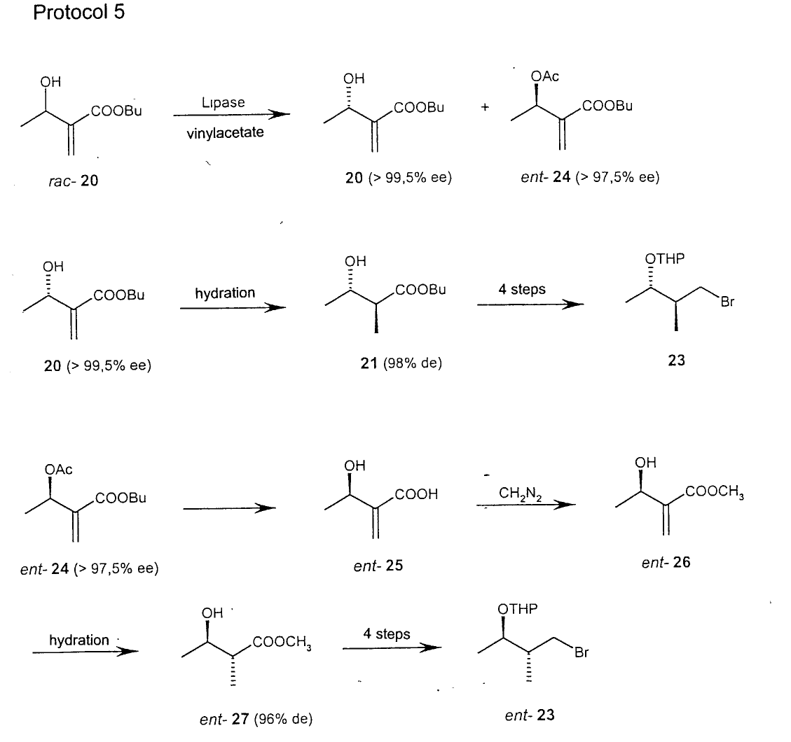

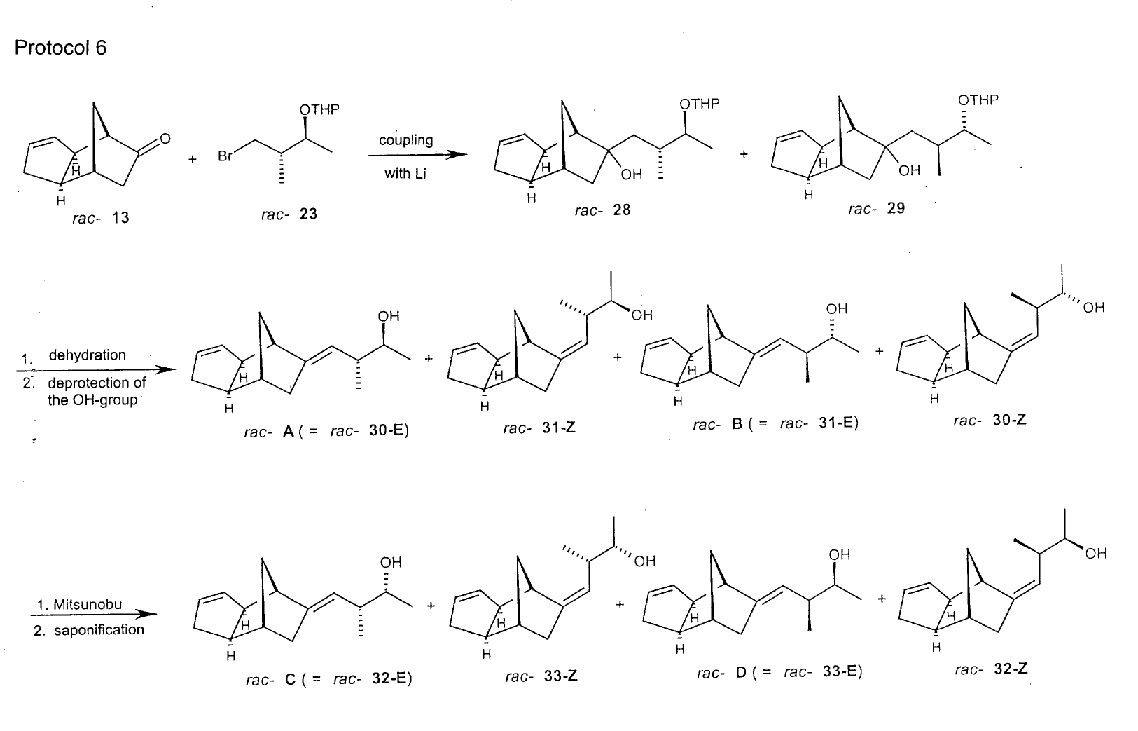

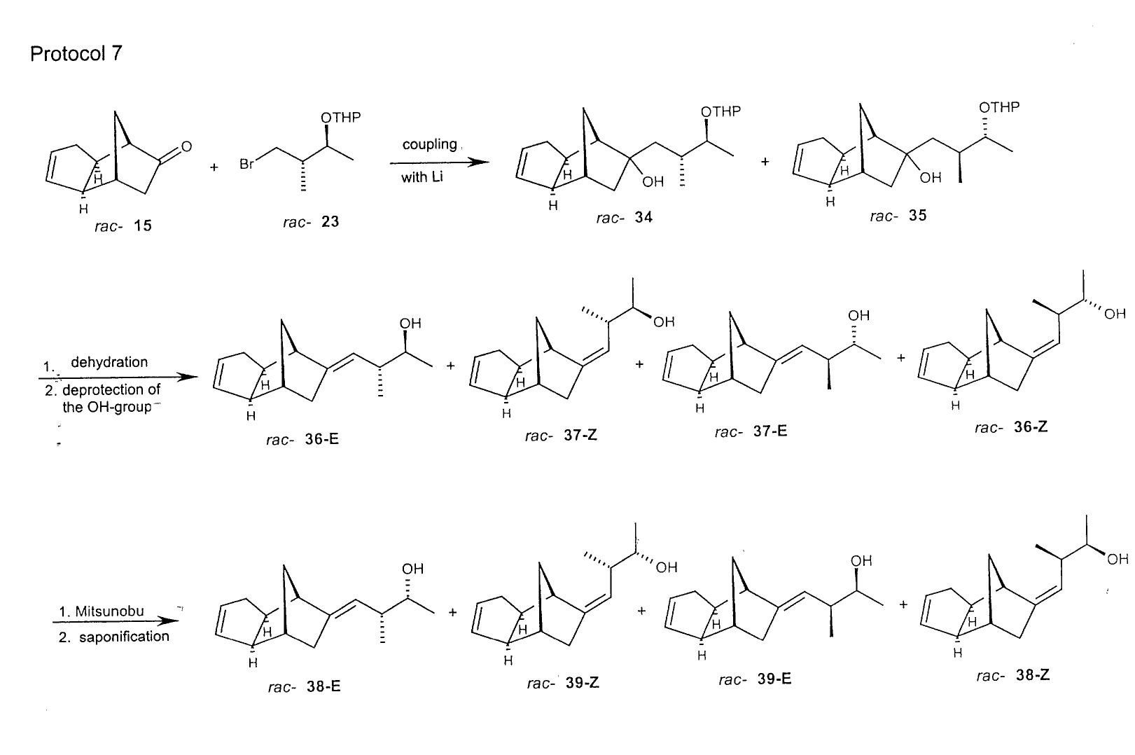

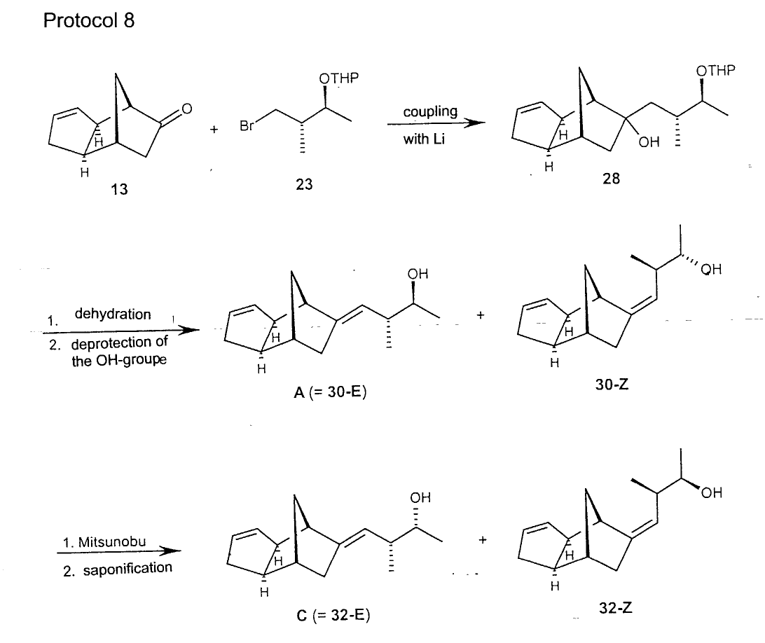

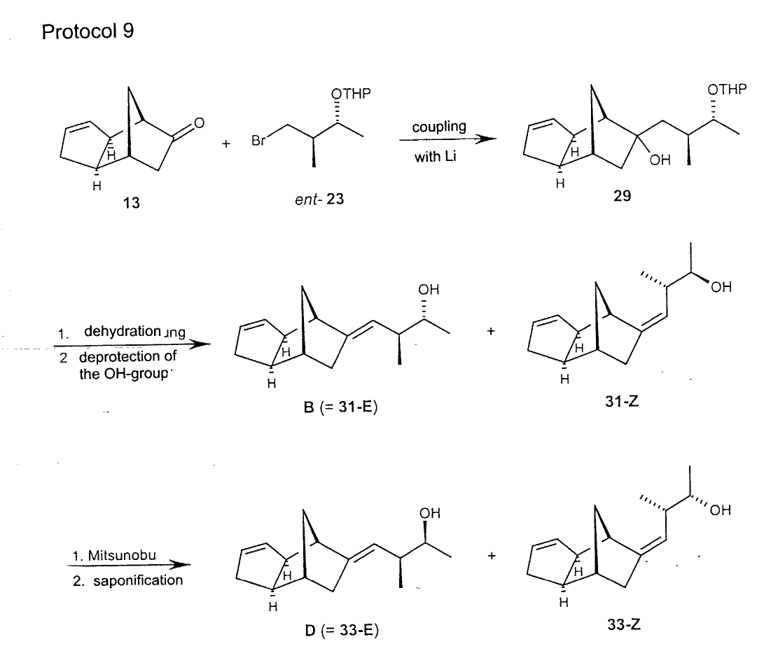

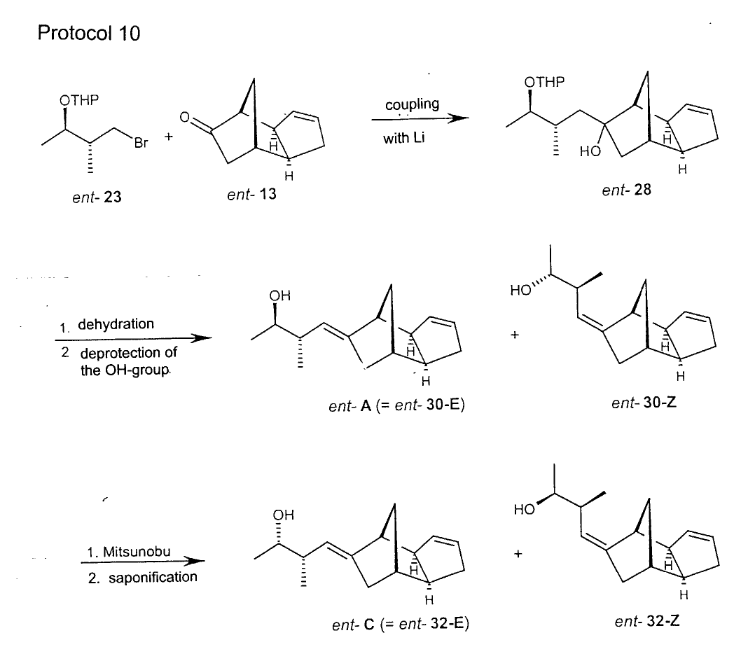

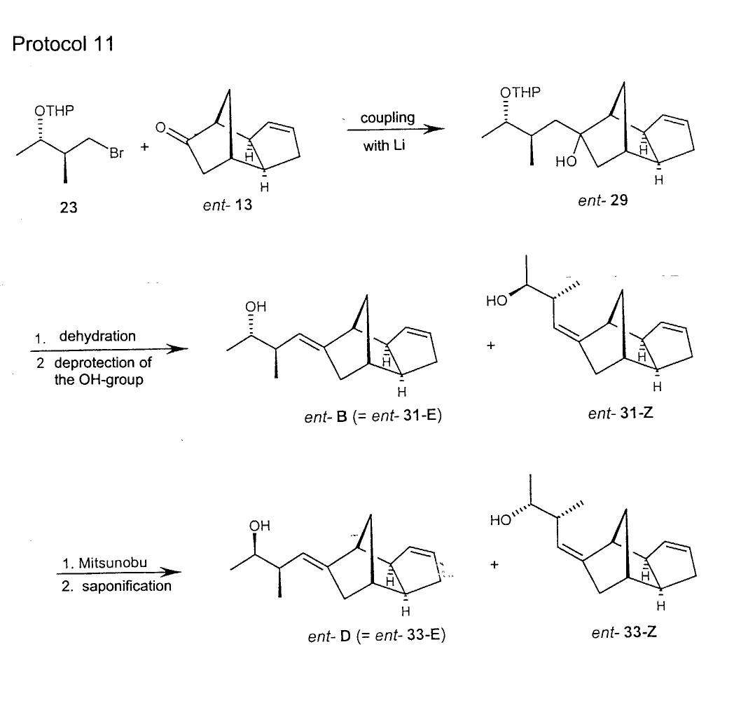

(2R/S, 3R/S)-3-Methyl-4-[(E,1R/S,2R/S,6R/S,7R/S)tricyclo[5.2.1.0^{2,6}]dec-4-en-8-ylidene]butan-2-ol,

wherein respectively independently from each other the following applies:

(2R/S, 3R/S) means (2R,3R), (2R,3S), (2S,3R) or (2S,3S) and

(E,1R/S,2R/S,6R/S,7R/S) means (E,1R,2R,6R,7R) or (E,1S,2S,6S,7S).

This compound is excellent for use as a fragrance or flavor compound.

### BACKGROUND OF THE INVENTION

Field of the Invention

The invention concerns new substances of the formula (2R/S, 3R/S)-3-methyl-4-[(E,1R/S,2R/S,6R/S,7R/S)tricyclo[5.2.1.0^{2,6}]dec-4-en-8-ylidene]butan-2-ol,

wherein respectively, independently from each, other the following applies:

(2R/S, 3R/S) means (2R,3R), (2R,3S), (2S,3R) or (2S,3S) and

(E,1R/S,2R/S,6R/S,7R/S) means (E,1R,2R,6R,7R) or (E,1S,2S,6S,7S).

The invention further concerns preferred configurational isomers (diastereomers, enantiomers) of the above mentioned compound with particularly pronounced sensorial characteristics as well as suitable mixtures.

The invention further concerns fragance and/or flavor compositions (organoleptic compositions) which include a sensorially (organoleptic) active (effective) amount of the mentioned compound (inclusive of the associated configurational isomers) or an appropriate configuration isomer mixture.

The invention also concerns processes for modification of the sensorial characteristics of a fragrance or flavor composition, wherein one or more components of the fragrance or aromatic composition is mixed with a sensorially effective amount of the inventive compound (inclusive of the associated configurational isomers) or a corresponding or equivalent mixture.

In the perfume and flavoring arts there is in general a continuous need for a synthetic fragrance and flavor substance, which can be produced ecomonically with sufficient quality, which remains storage stable for long periods of time even in contact with other substances, and which exhibits the desired olfactory or, as the case may be, flavor characteristics. Fragrances should have a pleasant fragrance note as close as possible to natural and should exhibit sufficient intensity and be able to favorably influence the fragrance of cosmetic or industrial goods advantageously. Flavor substances should have good biocompatability, should suggest typical taste components of known foods or even be identical thereto and should be capable of enhancing or supplementing the flavor of consumables, or to positively influence orally administered medicaments or the like. The discovery of fragrance and flavor substances which satisfy these requirements has until now required extensive investment and as a rule requires a large amount of experimentation, in particular when interesting new fragrance notes or flavor directions are desired.

The search for suitable fragrance of flavor substances is made difficult for the person skilled in the art by the following factors:

in mechanisms of fragrance or, as the case may be, flavor recognition is not know.

an objective-quantative characterization of fragrance or flavors is not possible.

the connection between the fragrance and/or flavor recognition on the one hand and a chemical structure of the fragrance and/or flavor substance on the other hand is not sufficiently understood.

frequently even small changes in the structure of known fragrance or flavor substances produce strong changes in the olfactory or as the case may be taste characteristics which leads to a comprise in bio-capability for the human organism.

The success of the search for suitable fragrance or flavor substances thus as a rule depends upon the intiution of the researcher.

### SUMMARY OF THE INVENTION

The present invention is concerned with the task, taking into consideration the above described general pre-requisites, of providing fragrance and flavor substances which are particularly suitable for providing conventional fragrance or flavor basic compositions with a scent evocative of natural sandalwood, or to modify the exising scent of these compositions in a desirable manner.

The substances to be provided should allow the perfumist or flavorist, when composing fragrances or flavors, a multifacetted employable alternative to the conventionally employed or known fragrances with sandalwood or sandalwood like fragrances. In the creative process (a lengthy process which, as a rule, is carried out by specialists) it is not the case that the composer simply envisions a pre-imagined existing fragrance or flavor, and then combines known fragrance or flavor substances by selection from a pattern or template in which specific fragrance or flavor aspects have been assigned in the literature. The fragrance or flavor characteristic of a composition cannot be precisely predicted in the sense of its addition, when only the individual components of the composition are known, since these components once mixed undergo unpredictable modifications. Thus, the compatability of the fragrance or flavor substance with the remaining components of a composition and the presence or absence of accompanying sensorial perceivable aspects is also of importance, in that these could influence the overall character of the finished composition, without this effect being predictable from the basic description of the pure substance.

The invention wais made based on the surprising recognition, that the inventive compound (2R/S, 3R/S)-3-methyl-4-(E,1R/S,2R/S,-6R/S,7R/S)tricyclo-[5.2.1.0^{2,6}]dec-4-en-8-ylidene]butan-2-ol according to the following Formula rac-1 (representation without specifying the absolute stereochemistry; rac═racemic) and its configurational isomers according to the following Formulas A , ent-A, B, ent-B, C, ent-C, D, ent-D, is particularly suitable for use as fragrance or flavor substances as well as for solving the above described problems.

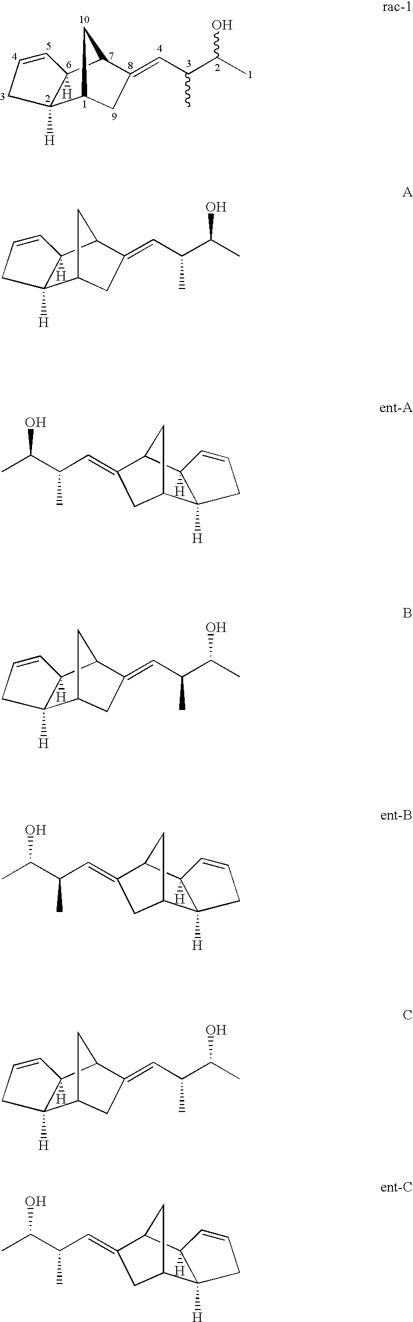

The above illustrated configurational isomers of the inventive compound respectively possess a very intense aroma, wherein however compounds with the configurational feature (E,1R,2R,6R,7R) exhibit a significant sandal-like aroma, which in some cases is colored by side aspects, and wherein the configurational isomer with the configuration feature (E,1S,2S,6S,7S) possess an aroma, which could more readily be described as woody.

Among the configurational isomers of the group of configurational isomers (E,1R,2R,6R,7R), which can be mentioned as being of particular interest, there is of further interest the compound of the configuration features (2S, 3R), that is, the compound according to Formula A, or (2S,3S), that is, the compound according to Formula D, because of its surprising side notes which is of particular significance for the creation of other fragrance compositions. This is described in greater detail below in the examples.

The inventive compound (represented by its 8 configurational isomers) belongs to a larger group of compounds, which are described in WO 86/03737. The compound 9-(2-methyl-3-hydroxy-butylidene)-2,6-exo-tricyclo[5.2.1.0^{2,6}]-decen-3(4) disclosed in Example 2 of this document includes overall 32 isomers, which can differ with respect to their position of the double bond at the terminal five-membered ring (carbon atoms C2 through C6), with respect to the configuration of the double bond between the C8 of the ring structure and the C4 of the side chain (E or Z-configuration) and with respect to its stereo chemistry (configuration at C2 and C3 in the side chain as well as configuration of the chiral centers in the ring structure). In WO 86/03737 there is however no mention of the corresponding isomers or even configurational isomers, rather there is only mention of the mentioned compound in its general form. Using the nomenclature as used otherwise in this text, the known compound which can be taken from WO 86/03737 is 3-methyl-4-[(E/Z,1RS,2RS,6RS,7RS)tricyclo[5.2.1.0^{2,6}]dec-4-en-8-ylidene]butan-2-ol and 3-methyl-4-[(E/Z,1RS,2SR,6RS,7RS)tricyclo[5.2.1.0^{2,6}]dec-3-en-8-ylidene]butan-2-ol (wherein it is presumed: E/Z means E or Z). Surprisingly it has been found, that among the 32 isomers of the compound known from WO 86/03737 only the inventive substance is of sensory relevance. The 24 total compounds of the Formula 3-methyl-4-[(E,1R/S,2S/R,6R/S,7R/S)-tricyclo[5.2.1.0^{2,6}]dec-3-en-8-ylidene]-butan-2-ol (Formula rac-2)

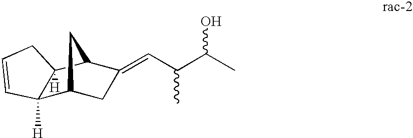

3-methyl-4-[(Z,1R/S,2R/S,6R/S,7R/S)tricyclo[5.2.1.0^{2,6}]dec-4-en-8-ylidene]-butan-2-ol (Formula rac-3)

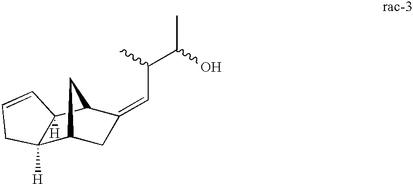

and

3-methyl-4-[(Z,1R/S,2S/R,6R/S,7R/S)tricyclo[5.2.1.0^{2,6}]dec-3-en-8-ylidene]-butan-2-ol (Formula rac-4)

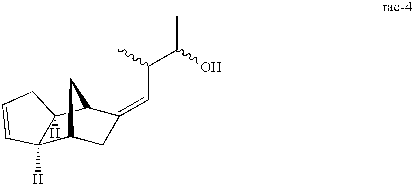

which could likewise be considered to be within the overall scope of the compound known in WO 86/03737, are sensorially uninteresting. If the person of ordinary skill would thus, for example for purposes of modification of a fragrance composition, not be directed to or aware of the fragrance compositions of the present invention, and instead were to add the mixture of isomers which for practical reasons would be the economical available form, then he would have the concern, that the sensorially characteristics of the mixture of the mixture would be primarily determined by the configurational isomers of the inventive compounds, rather than the inventive compounds. It is necessary that compounds out of the group of 24 sensorial uninteresting isomers (see above) must be suppressed from a sensorial aspect into the background. In particular, in the case of employment of the inventive configurational isomers with the configurational characteristic (E, 1R, 2R, 6R, 7R), which are characterized by their sandal-like fragrance, these allow the above mentioned fragrance compositions to be sensorially modified very effectively. It should also be noted that the process for modification disclosed in Examples 8 and 10 of WO 86/03737, involving use of the compound (including its total of 32 configurational isomers) 3-methyl-4-[E/Z-tricyclo[5.2.1.0² 6]dec-3/4-en-8-ylidene]butan-2-ol, does not suggest a modification which is first achieved by the use of the sensorially active amount of an inventive compound with the configurational features (E, 1R, 2R, 6R, 7R).

The invention is described in the following on the basis of examples.

### EXAMPLE 1

### Example of Synthesis Steps (see accompanying Protocol 1-11)

1.1 Synthesis of Cyclic Building Blocks

Alcohol Mixture rac-61rac-7 (Protocol 1)

The synthesis occurs starting with endo-dicyclopendadiene (rac-5) according to H. A. Bruson, T. W. Riener (J. Am. Soc. Chem. 67, 723 (1945))

Acetal Mixture rac-81rac-9 (Protocol 1)

To 75 g (0.5 mol) alcohol mixture rac-6/rac-7, 150 ml toluol and 6 g sodium carbonate were added with stirring and backflow 60 g (0.59 mol) acidic acicacidanhydrid. After 1 hour the batch was heated to 80° C. with addition of 100 g water and further stirred for 15 min. The aqueous phase was separated; the organic phase was washed neutral with soda solution and freed of toluol in a rotary evaporater. 94 g of acetatol mixture remained, which according to ¹H-NMR-spectrum contained the isomers rac-8 and rac-9 in the ratio 3:2.

Spectral data for the pure isomers is given below.

Oxoacetate rac-10 and rac-11 (Protocol 1)

To a solution of 156.7 g (0.816 mol) of acetal mixture rac-8/rac-9 in 1 liter acetic acid and 500 ml acetic acid and hydride was added with stirring 395 g (2.438 mol) sodium chromate. The resulting solution was stirred 3 days at 30-35° C. The reaction material was taken up or dissolved in 500 ml toluol and 3 liter water; the organic phase was washed with soda solution to neutral and freed of solvent at 15 hPa. There remained 165 g raw material, which was distilled using a 30 cm-vigreux column; after an initial discharge of 59.3 g of acetate mixture rac-8/rac-9 there was collected within the boiling point range 110-118° C. at 0.5 hPa 84.6 g of oxo-acetal mixture rac-1/rac-11 as a colorless fluid, indicated that the isomers rac-10 and rac-11 were obtained in the ratio 55:45. Following renewed distillation using a 1 m-rotating band column a fraction (48.3 g) was obtained, which comprised the lower boiling isomer rac-10 at 93% according to GC-analysis; while the isomer rac-11 remained enriched to 95% in the residue (33.2 g); both materials solidified upon cooling. Further crystallization out of tert-butyl-methylether produced pure rac-10 with a melting point of 119-119.5° C. and pure rac-11 with a melting point of 84.5-85.5° C. as colorless crystals.

Acetic Acid-(1RS,2RS,6SR,7RS,8SR)-3-oxotricyclo[5.2.1.0^{2,6}]dec-4-en-8-ylester (rac-10):

¹H-NMR (300 MHz, CDCl₃): δ=1.06 (dm, J=11 Hz, 1H), 1.36 (dm, J=11 Hz, 1H), 1.57 (ddd, J=13.5, 4.25, 2.75 Hz, 1H), 1.86 (ddd, J=13.5, 7, 2.5 Hz, 1H), 2.04 (s, 3H), 2.12 (d, J=5 Hz, 1H), 2.34 (s, 1H), 2.48 (d, J=4.25 Hz, 1H), 2.75-2.81 (m, 1H), 4.75 (d, J=7 Hz, 1H), 6.32 (dd, J=5.5,1.5 Hz, 1H), 7.53 ppm (dd, J=5.5, 2.75 Hz, 1H).

¹³C-NMR (75 MHz, CDCl₃): δ=21.2 (q), 28.3 (t), 37.7 (d), 38.2 (t), 43.2 (d), 46.1 (d), 52.2 (d), 76.7 (d), 137.7 (d), 164.1 (d), 170.6 (s), 210.3 ppm (s).

MS: m/z (%)=206 (1, M⁺), 147 (11), 146 (100), 145 (14), 120 (17), 118 (19), 117 (18), 95 (47), 91 (17), 66 (11), 43 (38).

Acetic Acid-(1RS,2SR,6RS,7RS,8SR)-5-oxotricyclo[5.2.1.0^{2,6}]dec-3-en-8-ylester (rac-11):

¹H-NMR (300 MHz, CDCl₃): δ=1.09 (dm, J=11 Hz, 1H), 1.39 (dm, J=11 Hz,1H), 1.59 (ddd, J=13.5, 4, 2.5 Hz, 1H), 1.93 (ddd, J=13.5, 7, 2.5 Hz, 1H), 2.02 (s, 3H), 2.14 (d, J=5 Hz, 1H), 2.30 (d, J=4.5 Hz, 1H), 2.47 (s, 1H), 2.70-2.76 (m,1H), 4.71 (d, J=7 Hz, 1H), 6.31 (dd, J=5.5, 1.5 Hz, 1H), 7.55 ppm (dd, J=5.5, 2.5 Hz, 1H).

¹³C-NMR (75 MHz, CDCl₃): δ=21.2 (q), 28.2 (t), 36.9 (d), 39.3 (t), 43.7 (d), 48.4 (d), 49.9 (d), 75.6 (d), 137.6 (d), 165.2 (d), 170.4 (s), 209.9 ppm (s).

MS: m/z (%)=206 (7, M+), 164 (29), 146 (100), 136 (29), 135 (21), 123 (30), 119 (24), 118 (21), 117 (27), 94 (28), 91 (30), 43 (45).

Acetate rac-8 (Protocol 1)

a) To a solution of 14.75 g (0.071 mol) oxo-acetate rac-11 and 200 g ethanol (96 Vol.-%) was added a spatula tip of copper(1)-iodide, and then with stirring and cooling to 20-25° C. there was portionwise added over 30 min 1.86 g (0.049 mol) sodium borohydride. The combination was stirred for 45 min and then after careful addition of 15% hydrochlorlic acid was hydrolized and neutralized. After filtation the ethanol was distilled away using a rotating evaporator; the remainder was added to diethylether and water. The aqueous phase was separated; the organic phase was washed with water and freed of solvent using a rotating evaporator. There remained 14.25 g acetic acid-(1RS,2RS,6RS,7RS,8SR)-5-hydroxytricyclo[5.2.1.0^{2,6}]dec-8-ylester (rac-12); according to GC-analysis no educt remained. MS (main isomer): m/z (%)=210 (peak, M⁺), 150 (37), 132 (42), 121 (29), 109 (32), 106 (100), 105 (29), 91 (34), 79 (43), 67 (34), 66 (26), 43 (60).

b) 8.9 g (0.078 mol) methansulfonic acid chloride was added to a solution of 14 g (0.066 mol) of the produced hydroxyacetate and 1.2 g 4-dimethyl-aminopyridine in 40 ml of absolute dichlormethane and 14 ml absolute pyridine at 0-5° C. with stirring, and stirred over night at 20° C. The product was added to 50 ml water, stirred 1 hour and then extracted with dichlormethane. The organic phase was washed with 10% sulfuric acid, neutralized with washing with soda solution and freed of solvent with a rotating evaporator. There remained 19 g of raw methanesulfonic acid ester of acetic acid-(1RS,2RS,6RS,7RS,8SR)-5-hydroxytricyclo[5.2.1.0^{2,6}]dec-8-ylester; according to DC-analysis (cyclohexane/ethylacetate 4:1) no educt remained.

c) To a solution of 14.4 g (0.05 mol) of the mesylate (methane sulfonate) in 150 ml absolute xylo (isomer mixture), 1 g dried lithium bromide and 15 g 1.8-diazabicyclo[5.4.0]undec-7-ene was added, with stirring for 3 hours with reflux. The reaction substance was washed with water and with 10% sulfuric acid and then neutralized with soda solution. Xylol was distilled away using a 20 cm-vigreux column. The residue (9.4 g), which according to DC-analysis (cyclohexane/ethylacetate 4:1) no longer contained educt any, was distilled in a micro-vigreux column. At 114-115° C. and 2 hPa 7.2 g acetate rac-8 were collected as colorless fluid.

Acetic acid-(1RS,2RS,6RS,7RS,8SR)tricyclo[5.2.1.0^{2,6}]dec-4-en-8-ylester (rac-8):

¹H-NMR (300 MHz, CDCl₃): δ=1.315 (br. s, 2H), 1.39 (ddd, J=13.5, 4, 2.5 Hz, 1H), 1.76 (dd, J=13.5,7 Hz, 1H), 1.89 (dm, J=17.5 Hz, 1H), 1.97-2.09 (m, 2H), 2.005 (s, 3H), 2.115 (s, 1H), 2.50-2.62 (m, 2H), 4.66 (dd, J=7, 2.5 Hz, 1H), 5.40-5.46 (m, 1H), 5.66-5.72 ppm (m, 1H).

¹³C-NMR (75 MHz, CDCl₃): δ=21.4 (q), 28.8 (t), 39.1 (t), 39.3 (t), 41.9 (d), 42.9 (d), 45.9 (d), 50.9 (d), 77.5 (d), 130.9 (d),132.7 (d), 170.7 ppm (s).

MS: m/z (%)=192 (40, M⁺), 124 (81), 117 (62), 105 (30), 91 (45), 83 (52), 82 (47), 67 (48), 66 (100), 43 (65).

Ketone rac-13 (Protocol 1)

The production of ketone rac-13 occured analogously to the below described production of ketone rac-15, that is, by saponification of acetate rac-8 or p-nitrobenzoate rac-16 to alcohol rac-6 followed by oxidation.

(1RS,2RS,6RS,7RS)tricyclo[5.2.1.0^{2,6}]dec-4-en-8-on (rac-13):

¹H-NMR (300 MHz, CDCl₃): δ=1.52 (dm, J=10.5 Hz, 1H), 1.655 (dm, J=10.5 Hz, 1H), 1.87 (dd, J=17.5,4 Hz, 1H), 1.99-2.10 (m, 2H), 2.33-2.42 (m, 3H), 2.71 (ddm, J=17.5, 9.5 Hz, 1H), 2.90-2.97 (m, 1H), 5.42-5.48 (m, 1H), 5.76-5.81 ppm (m, 1H).

¹³C-NMR (75 MHz, CDCl₃): δ=31.0 (t), 39.6 (t), 41.7 (d), 42.3 (d), 45.3 (t), 50.1 (d), 54.0 (d), 129.4 (d), 133.4 (d), 217.5 ppm (s).

MS: m/z (%)=148 (100, M+), 105 (35), 104 (26), 92 (38), 91 (57), 79 (49), 78 (28), 77 (32), 67 (21), 66 (35).

Acetate rac-9 (Protocol 1)

The production of acetate rac-9 occured analogously to the above described synthesis of acetate rac-8, that is from oxoacetate rac-10 via acetic acid (1RS,2RS,6RS,7RS,8SR)-3-hydroxy-tricyclo[5.2.1.0^{2,6}]dec-8-ylester (rac-14).

Acetic Acid-(1RS,2SR,6RS,7RS,8SR)tricyclo[5.2.1.0^{2,6}]dec-3-en-8-ylester (rac-9):

¹H-NMR (300 MHz, CDCl₃): δ=1.33 (br. s, 2H), 1.44 (ddd, J=13.5, 4.25, 2.5 Hz, 1H), 1.81 (ddd, J=13.5,7,2 Hz, 1H), 1.92 (dm, J=17 Hz, 1H), 2.005 (s, 3H), 2.02-2.16 (m, 3H), 2.535 (br. d, J=7.5 Hz, 1H), 2.62 (ddm, J=17, 10 Hz, 1H), 4.61 (dd, J=7, 2.5 Hz, 1H), 5.42-5.47 (m, 1H), 5.65-5.71 ppm (m, 1H).

¹³C-NMR (75 MHz, CDCl₃): δ=21.4 (q), 28.9 (t), 38.9 (d), 39.15 (t), 39.2 (t), 39.5 (d), 48.3 (d), 54.9 (d), 76.9 (d), 131.9 (d), 132.3 (d), 170.8 ppm (s).

MS: m/z (%)=192 (1, M⁺), 132 (48), 117 (22), 104 (12), 91 (21), 79 (14), 77 (15), 67 (31), 66 (100), 43 (42), 39 (14).

Ketone rac-15 (Protocol 1)

a) A solution of 9.6 g (0.05 mol) acetate rac-9 in 50 g methanol was stirred with addition of 5 g 50% sodium hydroxycide solution for 2 h with back flow or reflux. The methanol was distilled off using a rotating evaporator; the residue was taken up in 50 ml water and extracted with diethylether. The organic phase was washed with water and freed of solvent using a rotating evaporator. There remained 7.3 g alcohol rac-7.

MS: m/z (%)=150 (32, M⁺), 132 (13), 117 (18), 106 (27), 91 (39), 83 (20), 79 (26), 78 (21), 77 (24), 67 (46), 66 (100), 39 (24).

b) A solution of 1.5 g (0.01 mol) of the alcohol in 100 ml dichlormethane was stirred with 4.5 g pyridinium dichromate overnight at 20-25° C. The reaction product was filtered over silica gel; the filtrate was freed of solvent using a rotating evaporator and distilled at 1 hPa in a spherical flask. According to GC-analysis no educt remained. There remained 1.3 g ketone rac-15.

(1RS,2SR,6RS,7RS)tricyclo[5.2.1.0^{2,6}]dec-3-en-8-on (rac-15):

¹H-NMR (300 MHz, CDCl₃): δ=1.53 (dm, J=10.5 Hz, 1H), 1.745 (dm, J=10.5 Hz, 1H), 1.92 (dd, J=17.5,4 Hz, 1H), 1.98-2.14 (m, 2H), 2.33 (br. s, 1H), 2.40-2.49 (m, 2H), 2.70 (ddm, J=17.5, 10 Hz, 1H), 2.85-2.92 (m, 1H), 5.55-5.60 (m, 1H), 5.76-5.81 ppm (m, 1H).

¹³C-NMR (75 MHz, CDCl₃): δ=31.1 (t), 37.9 (d), 38.8 (t), 39.7 (d), 44.9 (t), 54.4 (d), 56.2 (d), 131.6 (d), 133.1 (d), 217.3 ppm (s).

MS: m/z (%)=148 (100, M⁺), 105 (28), 104 (34), 92 (30), 91 (55), 82 (52), 79 (48), 78 (29), 77 (34), 66 (63).

p-Nitrobenzoate rac-16 and rac-17 (Protocol 2)

75 g (0.5 mol) alcohol mixture rac-6/rac-7 was dissolved in 250 ml absolute pyridine, and 115 g (0.62 mol) 4-nitrobenzoic acid chloride was added in portions with stirring at 0-5° C. over 30 minutes, followed by stirring overnight at 20° C. To the product was added 300 g water; this was stirred for 1 hour and then taken up in 300 ml toluol. The aqueous phase was separated, the organic phase was twice washed with 150 g 10% sulfuric acid, neutralized with soda solution and freed of toluol in the rotating evaporator. There remained 149 g raw product as amorphis solid, which according to ¹H-NMR-spectrum contained the isomers rac-16 and rac-17 in the ratio 59:41. By repeated fractionation crystallization from tert.-butyl-methylether the main component and preferred crystallized isomer rac-16 was separated from isomer rac-17, which became concentrated in the mother liquor. There was obtained a pure rac-16 with a melting point 134-134.5° C. as a colorless powder.

4-nitrobenzoic Acid-(1RS,2RS,6RS,7RS,8SR)tricyclo[5.2.1.0^{2,6}]dec-4-en-8-ylester (rac-16):

¹H-NMR (300 MHz, CDCl₃): δ=1.43 (“tm”, J=11.5 Hz, 2H), 1.59 (ddd, J=13.5, 4, 2.5 Hz, 1H), 1.86-1.99 (m, 2H), 2.08-2.20 (m, 2H), 2.30 (br. s, 1H), 2.54-2.71 (m, 2H), 4.96 (dd, J=7, 2.5 Hz, 1H), 5.44-5.50 (m, 1H), 5.70-5.76 (m, 1H), 8.18 (dm, J=9 Hz, 2H), 8.27 ppm (dm, J=9 Hz, 2H).

¹³C-NMR (75 MHz, CDCl₃): δ=29.1(t), 39.2 (t), 39.3 (t), 41.9 (d), 42.9 (d), 46.0 (d), 50.8 (d), 79.2 (d), 123.4 (2d), 130.6 (2d), 130.7 (d), 132.9 (d), 136.1 (s), 150.4 (s), 164.3 ppm (s).

Alcohol 6 and Acetate ent-8 by Enzymatic Racemate Cleavage of Acetate rac-8 (Protocol 3)

5 g lipase (Porcine Pancreas Type II; product of Sigma) was prepared by stirring in 430 ml phosphate-buffer (pH=7). After 30 minutes the suspension was adjusted with 1 N sodium solution or, as the case may be, diluted hydrochloric acid. Subsequently 11.50 g (60 mmol) acetate rac-8 were added dropwise and the suspension was stirred 11 days at 20-25° C. During this time the suspension was adjusted to pH=7 at regular intervals with 1 N sodium solution and subsequently a total of 11 g lipase was added. The transformation or conversion was monitored by gas chromatography and interrupted upon reaching the ratio of alcohol to acetate of 55:45. After addition of 500 ml water it was extracted three times with respectively 300 ml methyl-tert-butylether. The recombined organic phases were washed with 5% sodium chloride solution and then dried over sodium sulfate. After removal of the solvent in vacuum the obtained raw product was chromatographed on silica gel 60 (product of Merck; cyclohexane/ethyl acetate 95:5 to 80:20). There was obtained 5.2 g (60% ee) enantiomer enriched acetate ent-8 and 4.9 g (48% ee) enantiomer enriched alcohol 6. The enantiomer surplus (ee) of 6 was determined according to chiral gas chromatography (precolumn: DBWax, 30 m, 80-240° C. 4° C./min; main column: Ethyl-β-bicchi, 15 min 80° C., −200° C. 1C/min): t_{R}=30.1 (74% 6), 30.3 min (26% ent-6).

(1 R,2R,6R,7R,8S)tricyclo[5.2.1.0^{2,6}]dec-4-en-8-ol (6):

¹H-NMR (300 MHz, CDCl₃): δ=1.19-1.32 (m, 2H), 1.38 (ddd, J=10.1, 1.4, 1.4 Hz, 1H), 1.67 (ddd, 13.0, 7.0, 2.4 Hz, 1H), 1.87 (dm, J=17.2 Hz, 1H), 1.96 (br. d, J=1.3 Hz, 2H), 1.98-2.05 (m,1H), 2.40-2.47 (m, 1H), 2.53 (ddddd, J=17.0, 9.6, 2.2, 2.2, 1.3 Hz, 1H), 3.05 (br. s, OH), 3.79 (d, J=6.6 Hz,1H), 5.42 (dddd, J=4.6, 2.2, 2.2, 2.2 Hz,1H), 5.66 ppm (dddd, J=5.9,2.0, 2.0,2.0 Hz, 1H).

¹³C-NMR (75 MHz, CDCl₃): δ=28.0 (t), 39.2 (t), 41.7 (t), 42.0 (d), 42.9 (d), 48.7 (d), 51.2 (d), 74.5 (d), 131.2 (d), 132.2 ppm (d).

MS: m/z (%)=150 (45, M+), 132 (38), 117 (78), 105 (31), 91 (60), 83 (42), 79 (30), 77 (32), 67 (34), 66 (100).

Alcohol ent-6 (Protocol 3)

5.2 g (27 mmol) of the enantiomer enriched acetate ent-8 was heated with reflux in 5 ml 50% sodium hydroxide solution and 35 ml methanol for 2 hours with stirring. After addition of 100 ml water it was extracted three times with respectively 50 ml diethylether. The recombined organic phases were washed with 5% sodium chloride solution and dried over sodium sulfate. The solvent was removed under reduced pressure in a rotating evaporator and the obtained raw product was chromatographed using silica gel 60 (producer Merck; Cyclohexane/Ethylacetate 9:1 to 8:2). One obtains 4.06 g (60% ee) enantiomer enriched alcohol ent-6.

The enantiomer surplus of ent-6 was determined per chiral GC (see above): t_{R}=30.1 (20% 6), 30.3 min (80% ent-6).

(1 S,2S,6S,7S,8R)Tricyclo[5.2.1.0^{2,6}]dec-4-en-8-ol (ent-6):

The technical data correspond to that of alcohol 6.

Camphor Acid Ester ent-19 (Protocol 3)

To a solution of 4 g (27 mmol) of the enantiomer enriched alcohol ent-6 (60% ee) in 40 ml pyridine was added 5.96 g (28 mmol) (S)-(−)-Camphor acid chloride (ent-18) as well as 40 mg 4-dimethyl amino pyridine and stirred 24 hours at 20-25° C. After addition of 100 ml water and acidification with 20% sulfuric acid it was extracted three times with respectively 50 ml diethyl-ether. The combined organic phases were washed with 5% soda solution as well as 5% sodium chloride solution and subsequently dried over sodium sulfate. After removal of the solvent with a rotating evaporator the obtained diastereomer mixture was recrystallized out of diethyl ether, by which crystalliztion there was enriched the preferred main diastereomer ent-19 (the diastereomer content was checked using the ¹³C-NMR-signal which was significant for the side diastereomer of 78.85 or as the case may be 167.0 ppm). By repeated crystallization one obtained 4.31 g pure camphor acid ester ent-19 as colorless needles with melting point 127.5-128° C.

The absolute configuration of ent-19 was determined using x-ray crystalagraphic analysis of a crystal.

(1S,4R)-3-Oxo-4,7,7-trimethyl-2-oxabicyclo[2.2.1 ]heptan-1-carbonic acid-(1S,2S,6S,7S,8R)tricyclo[5.2.1.0^{2,6}]dec-4-en-8-ylester (ent-19):

¹H-NMR (300 MHz, CDCl₃): δ=0.96 (s, 3H), 1.05 (s, 3H), 1.11 (s, 3H), 1.34 (s, 2H), 1.48 (ddd, J=13.7, 4.2, 2.2 Hz, 1H), 1.67 (ddd, J=13.7, 4.2, 2.2 Hz, 1H), 1.77-2.00 (m, 7H), 2.41 (ddd, J=13.3, 10.5, 4.2 Hz, 1H), 2.50-2.70 (m, 2H), 4.83 (dd, J=7.0, 2.2 Hz, 1H), 5.44 (dddd, J=5.1, 2.4, 2.4,2.4 Hz,1H), 5.70 ppm (dddd, J=5.9,2.0,2.0,2.0 Hz, 1H). ¹³C-NMR (75 MHz, CDCl₃): δ=9.7 (q), 16.7 (q), 16.8 (q), 28.85 (t), 28.9 (t), 30.5 (t), 39.0 (t), 39.2 (t), 41.8 (d), 42.9 (d), 46.1 (d), 50.7 (d), 54.1 (s), 54.8 (s), 78.9 (d), 91.0 (s), 130.6 (d), 133.0 (d), 167.1 (s), 178.3 ppm (s).

Camphor Acid Ester 19 (Protocol 3)

Analogously to the production of ent-19, 2.32 g (15 mmol) enantiomer, enriched alcohol 6 (48% ee) was esterified with 3.4 g (16 mmol) (R)-(+)-camphor acid chloride (18). After preparation the obtained diastereomer mixture was recrystallized out of diethyl ether. By repeated crystallization 2.77 g pure camphor acid ester 19 was obtained as colorless needles with a melting point 127.5-128° C.

(1R,4S)-3-Oxo-4,7,7-trimethyl-2-oxabicyclo[2.2.1]heptan-1-carbonic Acid-(1R,2R,6R,7R,8S)tricyclo[5.2.1.0^{2,6}]dec-4-en-8-ylester (19):

The spectral data correspond to that of ester ent-19.

Ketone ent-13 (Protocol 3)

a) To a solution of 4.31 g (13 mmol) diastereomer pure camphor acid ester ent-19 in 50 ml methanol was added 5 g 50% sodium hydroxide solution and heated for 2 hours with stirring and reflux. After addition of 100 ml water it was extracted three times with respectively 50 ml diethyl ether. The combined organic phases were washed with 5% sodium chloride solution and subsequently dried over sodium sulfate. After removal of the solvent, there remained 1.95 g (>98% ee) raw alcohol ent-6.

The enantiomer surplus of ent-6 was determined per chiral GC (see above): t_{R}=30.1 (<1% 6), 30.3 min (>99% ent-6).

b) To a solution of 1.95 g of the present raw alcohol in 100 ml dichlormethane there was added with stirring in portions a total of 6.77 g (18 mmol) pyridiniumdichromate followed by stirring for 16 hours at 20-25° C. The reaction mixture was filtered over silica gel; the filtrate was freed of solvent using a rotating evaporator, the raw product was chromatographed on silica gel 60 (manufactuer Merck; cyclohexane/ethylacetate 9:1 to 8:2). There were obtained 1.74 g ketone ent-13.

(1S,2S,6S,7S,8R)Tricyclo[5.2.1.0^{2,6}]dec-4-en-8-on (ent-13):

The spectral data correspond to that of ketone rac-13.

Ketone 13 (Protocol 3)

Analogous to the production of ent-13 first 2.77 g (8.4 mmol) diastereomer camphor acid ester 19 were saponified. After the procedure there remained 1.25 g (>98% ee) raw alcohol 6. The enantiomer surplus of 6 was determined per chiral GC (see above): t_{R}=30.1 (>99% 6), 30.3 min (<1% ent-6).

Then 1.2 g of the present raw alcohol was oxidized with 3.76 g pyridiniumdichromate (10 mmol). After processing and chromotographography on silica gel 1.03 g ketone 13 were obtained.

(1R,2R,6R,7R,8S)tricyclo[5.2.1.0^{2,6}]dec-4-en-8-on (13):

The spectral data corresponded to that of ketone rac-13.

1.2 Synthesis of the Side Chain Building Blocks

3-Hydroxy-2-methylene-butyric Acid Butyl Ester (rac-20) (Protocol 4)

The preparation occurred according to S. E. Drewes, N. D. Emslie (J. Chem. Soc., Perkin Trans. 1, 2079 (1982)).

3-Hydroxy-2-methylene-butyric Acid Butyl Ester (rac-20):

¹H-NMR (300 MHz, CDCl₃): δ=0.955 (t, J=7.5 Hz, 3H), 1.355 (d, J=6.5 Hz, 3H), 1.36-1.48 (m, 2H), 1.62-1.72 (m, 2H), 3.53 (br. s, 1H), 4.175 (t, J=6.5 Hz, 2H), 4.62 (q, J=6.5 Hz, 1H), 5.855 (t, J=1.25 Hz, 1H), 6.20 ppm (dd, J=1.25, 0.75 Hz, 1H).

¹³C-NMR (75 MHz, CDCl₃): δ=13.7 (q), 19.3 (t), 22.5 (q), 30.7 (t), 64.7 (t), 66.6 (d), 123.6 (t), 144.4 (s), 166.7 ppm (s).

MS: m/z (%)=157 (35, M+-15), 101 (100), 99 (51), 98 (46), 83 (53), 73 (44), 55 (54), 43 (56), 41 (69), 29 (96), 27 (79).

(2RS,3RS)-3-Hydroxy-2-methyl-butyric Acid Butyl Ester (rac-21) (Protocol 4)

25 g (0.145 mol) 3-Hydroxy-2-methylene-butyric acid butyl ester (rac-20) was dissolved in 100 ml absolute dichloromethane and degassed with nitrogen. After addition of 0.1 g rhodium(I)-[1,4-bis-(diphenylphospino)-butane]-(2,5-norbornadiene)-tetrafluoroborate (product of Aldrich) the solution was stirred 3 hours at 0.5-1 MPa and 20-25° C. in a water vapor atmosphere. The reaction material was filtered over talcum powder, freed of solvent in a rotating evaporator and distilled under 1 hPa in a spherical flask. There remained 24.7 g product, which according to GC/MS-analysis contained the anti-ester rac-21 with 98% de.

(2RS,3RS)-3-Hydroxy-2-methyl-butyric Acid Butyl Ester (rac-21):

¹H-NMR (300 MHz, CDCl₃): δ=0.94 (t, J=7.5 Hz, 3H), 1.165 (d, J=7 Hz, 3H), 1.205 (d, J=6.5 Hz, 3H), 1.32-1.46 (m, 2H), 1.58-1.68 (m, 2H), 2.465 (dq, J=7,7 Hz, 1H), 3.26 (br. s, 1H), 3.905 (dq (5 Linien), J=6.5, 7 Hz, 1H), 4.11 ppm (t, J=6.5 Hz, 2H).

¹³C-NMR (75 MHz, CDCl₃): δ=13.7 (q), 13.8 (q), 19.2 (t), 20.5 (q), 30.7 (t), 47.2 (d), 64.4 (t), 69.3 (d), 175.9 ppm (s).

MS: m/z (%)=159 (4, M+-15), 130 (13), 103 (13), 101 (61), 74 (100), 73 (13), 57 (30), 56 (34), 45 (16), 41 (13).

Tetrahydropyranylether (rac-23) of (2RS,3RS)-4-Brom-3-methylbutane-2-ol (Protocol 4)

a) To a solution of 17.4 g (0.1 mol) (2RS,3RS)-3-hydroxy-2-methyl-butyric acid butyl ester (rac-21) in 100 ml absolute diethylether there was added with stirring at 20-25° C. p-toluol sulfonic acid, followed by 10 g (0.12 mol) 3,4-dihydro-2H-pyran. After 4 hours the reaction material was washed with sodium hydoxide solution and freed of solvent in a rotating evaporator. According to GC-analysis no educt remained. There remained 25.1 g raw tetrahydropyranylether of (2RS,3RS)-3-hydroxy-2-methyl-butyric acid butyl ester.

MS (exemplary diastereomer): m/z (%)=257 (trace, M⁺−1), 157 (11), 101 (63), 83 (10), 57 (16), 56 (10), 55 (9), 41 (10).

b) To a solution of 25 g of the present raw ester in 50 ml absolute tetrahydrofuran there was added, dropwise with stirring at 0-10° C., a suspension of 2.3 g (0.06 mol) lithium alanate in 200 ml absolute tetrahydrofuran. Then, stirring was carried out for 1 hour with reflux. The surplus of lithium alanate was carefully decomposed with water at 0° C.; then the residue was filtered over zeolite and freed of solvent in a rotating evaporator. According to GC-analysis no educt remained. The result was 17.9 g raw (2SR,3RS)-2-methyl-3-(tetrahdro-2H-pyran-2-yloxy)butan-1-ol (rac-22; Protocol 4).

MS (exemplary diastereomer): m/z (%)=187 (Peak, M⁺−1), 129 (7), 101 (19), 85 (100), 69 (12), 67 (10), 57 (13), 56 (25), 45 (12), 43 (10), 41 (16).

c) To a solution of 9.4 g of the present raw alcohol (rac-22) in 100 ml absolute toluol, 12.5 g triethylamine and 1 g N,N,N′,N′-tetramethyl-hexan-1,6-diamine there was added with stirring at 0° C. 12.5 g (0.06 mol) p-toluol sulfonic acid chloride, and stirring continued overnight at 20° C. The product was added to 100 g water with stirring for 1 hour. The aqueous phase was separated; the organic phase was washed with 10% sulfonic acid, washed to neutral with soda solution and freed of toluol in a rotating evaperator. According to DC-analysis (cyclohexan/eEthylacetate 4:1) no educt remained. There remained 17 g raw p-toluol sulfonic acid ester of (2SR,3RS)-2-methyl-3-(tetrahdro-2H-pyran-2-yloxy)butan-1-ol.

d) In a solution of 17 g of the present raw tosylate in 150 g absolute acetone, 17.4 g fresh dried lithium bromide, 2.5 g sodium carbonate and 5 g molecular sieve 4 A were refluxed with stirring and under exclusion of moisture for 3 hours. After cooling the residue was filtered over ziolite; to the filtrate was added 150 ml dichlormethane, filtered again over ziolite and freed of solvent in a rotating evaporator. According to DC-analysis (cCyclohean/ethylacetate 4:1) no educt remained. Spherical flask distillation of the raw product (12.2 g) at 1 hPa produced 10.5 g bromide rac-23.

According to NMR-analysis the two diastereomers tetrahydropyranylether (rac-23) of (2RS,3RS)-4-bromo-3-methylbutan-2-ol were present in a ratio of 1:1:

¹H-NMR (300 MHz, CDCl₃): δ=0.995 (d, J=7 Hz, 1.5H), 1.05 (d, J=7 Hz, 1.5H), 1.11 (d, J=6 Hz, 1.5H), 1.24 (d, J=6 Hz, 1.5H), 4.67 (dd, J=4.5,3 Hz, 0.5H), 4.71 ppm (dd, J=4, 3.5 Hz, 0.5H).

¹³C-NMR (75 MHz, CDCl₃): δ=14.9 (q), 15.3 (q), 15.9 (q), 18.6 (q), 18.8 (t), 19.7 (t), 25.5 (2t), 31.1 (2t), 38.2 (t), 38.6 (t), 40.8 (d), 41.4 (d), 62.6 (t), 63.0 (t), 72.5 (d), 77.0 (d), 95.2 (d), 100.5 ppm (d).

MS (representative or exemplary diastereomer): m/z (%)=251/249 (1/1, M⁺−1), 151/149 (5/5), 129 (13), 101 (19), 85 (100), 69 (38), 67 (10), 56 (18), 55 (11), 43 (11), 41 (23).

(S)-(−)-3-Hydroxy-2-methylen-butyric Acid Butyl Ester (20) and (R)-(+)-3-Acetoxy-2-methylen-butyric Acid Butyl Ester (ent-24) (Protocol 5)

The synthesis occurred according to K. Burgess, L. D. Jennings (J. Org. Chem. 55, 1138 (1990)). According to the chiral gas chromatography (precolumn: DBWax, 30 minutes, 80-240° C. 4° C./minute; main column: ethyl-β-bicchi, 15 minute 80° C., -200° C. 1° C./minute) the optical purity of the 3S-compound 20 was determined to be>99.5% ee and that of the 3R-compound ent-24 as>97.5% ee.

(2S,3S)-3-Hydroxy-2-methyl-butyric Acid Butyl Ester (21) (Protocol 5)

Produced by hydration of (S)-(−)-3-hydroxy-2-methyl butyric acid butyl ester (20), analogous to the above description.

Tetrahydropyranylether (23) of (2S,3S)-4-Brom-3-methylbutan-2-ol (Protocol 5)

The production occurred analogously to the above described, four step reaction sequence from (2S,3S)-3-hydroxy-2-methyl-butyric acid butyl ester (21).

(2R,3R)-3-Hydroxy-2-methyl-butyric acid methyl ester (ent-27) (Protocol 5)

a) 21.4 g (0.1 mol) (R)-3-acetoxy-2-methylene-butyric acid methyl ester (ent-24) are refluxed with a solution of 8.4 g sodium hydroxide in 90 g ethanol and 10 g water with stirring. The product was freed of alcohol in a rotating evaporator, taken up in 150 ml water and washed with diethylether. The aqueous phase was adjusted with 10% sulfuric acid to pH=2.5, saturated with common salt and then extracted with ethyl acetate. After reduction of the extract in a rotation damper there remained 11.1 g raw (R)-3-hydroxy-2-methylene-butyric acid (ent-25).

¹H-NMR (300 MHz, CDCl₃): δ=1.40 (d, J=6.5 Hz, 3H), 4.69 (q, J=6.5 Hz, 1H), 5.96 (s, 1H), 6.35 (s, 1H), 8.22 ppm (br. s, 2H).

¹³C-NMR (75 MHz, CDCl₃): δ=22.1 (q), 66.7 (d), 126,2 (t), 143.1 (s), 170.5 ppm (s).

b) To a solution of 11 g of the present raw acid in 200 ml absolute diethylether there was added at 0° C. with stirring 350 ml of a fresh produced ethereal diazomethane solution (approximately 0.12 mol CH₂N₂; produced according to T. J. de Boer, H. J. Backer in Org. Synth. 36,16 (1956)) followed by stirring for 15 minutes at 20° C. To the remainder was added 5 g acetic acid, followed by stirring 5 minutes and then taking up in 100 ml water. The aqueous phase was separated; the organic phase was washed to neutral with sodium hydroxide solution and freed of solvent in a rotation evaporator. Spherical flask distillation of the raw methylester (11.5 g) at 1 hPa produced 10.9 g (R)-(+)-3-hydroxy-2-methylene-butyric acid methylester (ent-26).

MS: m/z (%)=115 (73, M+-15), 98 (27), 87 (43), 83 (100), 55 (73), 45 (29), 43 (85), 29 38), 27 (55).

The present ester ent-26 (Protocol 5) was hydrated, analogously to the description for rac-20. According to GC/MS-analysis the thus produced product contained the anti-ester ent-27 with 96% de.

(2R,3R)-3-Hydroxy-2-methyl-butyric Acid Methylester (ent-27):

¹H-NMR (300 MHz, CDCl₃): δ=1.15 (t, J=7 Hz, 3H), 1.195 (t, J=6.5 Hz, 3H), 2.49 (dq, J=7, 7 Hz, 1H), 3.55 (br. s, 1H), 3.70 (s, 3H), 3.915 ppm (dq (6 Linien), J=6.5, 7 Hz, 1H).

¹³C-NMR (75 MHz, CDCl₃): δ=13.5 (q), 20.4 (q), 47.2 (d), 51.7 (q), 69.2 (d), 176.2 ppm (s).

MS: m/z (%)=117 (9, M⁺−15), 101 (19), 88 (100), 87 (15), 85 (21), 59 (12), 57 (52), 56 (25), 55 (16), 45 (24), 43 (12).

Tetrahydropyranylether (ent-23) of (2R,3R)-4-Brom-3-methylbutane-2-ol (Protocol 5)

The production occurred analogously to the above described four step reaction sequence for (2R,3R)-3-hydroxy-2-methyl-butyric acid methylester (ent-27).

1.3 Synthesis of Inventive Isomers and Comparative Isomers

Alcohol Mixture rac-A/rac-31-Z/rac-B/rac-30-Z (Protocol 6)

a) In a nitrogen atmosphere, to 87 mg (12.5 mmol) lithium (fine separated granulate) in 5 ml absolute tetrahydrofuran portionwise in 2 hours at 20-25° C. there was added with stirring a solution of 0.74 g (5 mmol) ketone rac-13 and 1.38 g (5.5 mmol) bromide rac-23 in 2.5 ml absolute tetrahydrofuran, and the stirring was continued overnight with constant temperature. The product was filtered of surplus lithium over glass wool then hydrolized with ice water and taken up in diethylether. The aqueous phase was separated, after combining the organic phases in the rotating evaporator there remained 1.6 g of the raw mixture of the alcohol rac-28 und rac-29.

MS (illustrative isomer): m/z (%)=302 (2, M⁺−18), 219 (14), 218 (13), 191 (22), 149 (12), 105 (11), 91 (10), 85 (100), 84 (10), 79 (11), 67 (24).

b) To a solution of 1.6 g of the present raw alcohol mixture in 25 ml absolute pyridine there was added with stirring at 0° C. 7.67 g phosphoroxychloride with stirring overnight at 20° C. The reaction product was carefully poured over ice water and then taken up in diethylether. The aqueous phase was separated; the organic phase was washed with 10% sulfuric acid, washed to neutral with soda solution and freed of solvent in a rotating evaporator. There remained 1.5 g raw THP-ether mixture of the alcohol rac-A/rac-31-Z/rac-B/rac-30-Z.

MS (examplary isomer): m/z (%)=302 (Peak, M⁺), 174 (5), 146 (5), 107 (10), 86 (5), 85 (100), 79 (7), 67 (17), 41 (5).

c) A solution of 1.5 g of the present raw THP-ether mixture in 15 ml absolute methanol, 1 g molecular 3 Å and 50 mg p-toluol sulfonic acid were stirred for 2 hours at 20-25° C. The reaction product was filtered, then added were 0.1 g sodium carbonite, and the methanol was distilled off in a rotating evaporator. The residue was taken up in water and dimethanol ether. The aqueous phase was separated, the organic phase was freed of solvent in a rotating evaporator. The obtained raw product (1.1 g) was chromatographed on silicia gel (producer Merck; Hexan/Diethylether 7:3); which resulted in 0.62 g alcohol mixture rac-A/rac-31-Z/rac-B/rac-30-Z.

Gas chromatograph (60 m HP5, 60-250° C. 4° C./min):

t_{R}=30.80 (rac-30-Z), 30.95 (rac-31-Z), 31.00 (rac-B=rac-31-E), 31.05 min (rac-A=rac-30-E).

The E/Z-ratio was determined by integration of the characteristic ¹H-NMR-signal for the side chain vinyl portion at 5.08 (E-isomer) and 4.85 ppm (Z-isomer) at 55:45.

NMR-analysis Produced

(2SR,3RS)-3-methyl-4-[(E,1RS,2RS,6RS,7RS)tricyclo[5.2.1.0^{2,6}]dec-4-en-8-ylidene]butan-2-ol (rac-A=rac-30-E) and (2RS,3SR)-3-methyl-4-[(Z,1 RS,2RS,6RS,7RS)tricyclo[5.2.1.0^{2,6}]dec-4-en-8-ylidene]butan-2-ol (rac-31-Z) as main isomers and rac-B (=rac-31-E) and rac-30-Z as minor isomers:

Spectral data can be seen under alcohol mixture A/30-Z and under alcohol mixture B/31-Z.

Alcohol Mixture rac-C/rac-33-Z/rac-D/rac-32-Z (Protocol 6)

a) To a solution of 0.1 g alcohol mixture rac-A/rac-31-Z/rac-B/rac-30-Z, 0.22 g triphenylphosphine and 0.06 g benzoic acid in 4 ml absolute tetrahydrofuran, there was added with stirring at 20° C. 0.1 g azodicarbonic acid diethylester, followed by stirring for 3 days at 20-25° C. The reaction product was taken up in water and diethylether. The aqueous phase was separated; the organic phase was washed with 10% sulfuric acid and with soda solution and freed of solvent in a rotating evaporator. The obtained raw product (0.45 g) was chromatographed in silica gel 60 (product of Merck; hexan/diethylether 7:3); there resulted 82 mg of a benzoate mixture of the alcohols rac-C/rac-33-Z/rac-D/rac-32-Z.

MS (exemplary isomer): m/z (%)=322 (peak, M⁺), 200 (38), 173 (22), 134 (27), 133 (100), 107 (26), 106 (16), 105 (85), 91 (17), 79 (12), 77 (31), 67 (32).

b) A solution of 82 mg of the present benzoate mixture in 3 ml methanol and 25 mg 50% sodium hydroxide solution were refluxed with stirring for 2 hours. The product was freed of methanol in a rotating evaporator; the residue was taken up in water and dichlormethane. The aqueous phase was separated; the organic phase was washed three times with water and freed of solvent in a rotating evaporator. There remained 52 mg alcohol mixture rac-C/rac-33-Z/rac-D/rac-32-Z.

Gas chromatograph (60 m HP5, 60-250° C. 4° C./min):

t_{R}=31.35 (rac-32-Z), 31.40 (rac-33-Z), 31.50 (rac-D=rac-33-E), 31.60 min (rac-C=rac-32-E).

The E/Z-ratio was determined by integration of the characteristic of ¹H-NMR-signal for the side chain-vinyl proton at 5.09 (E-isomer) and 4.86 ppm (Z-isomer) as being 55:45.

NMR-Analysis showed

(2RS,3RS)-3-methyl-4-[(E,1RS,2RS,6RS,7RS)tricyclo[5.2.1.0^{2,6}]dec-4-en-8-ylidene]butan-2-ol (rac-C=rac-32-E) und (2SR,3SR)-3-methyl-4-[(Z,1RS,2RS,6RS,7RS)tricyclo[5.2.1.0^{2,6}]dec-4-en-8-ylidene]butan-2-ol (rac-33-Z) as main isomer and rac-D (=rac-33-E) and rac-32-Z as side isomer:

Spectral data see alcohol mixture C/32-Z and alcohol mixture D/33-Z.

Alcohol Mixture rac-36-E/rac-37-Z/rac-37-E/rac-36-Z (Protocol 7)

Production occurred analogously to the above production of alcohol mixture rac-A/rac-31-Z/rac-B/rac-30-Z, that is, from ketone rac-15 and bromide rac-23 via coupling alcoholic mixture rac-34/rac-35 and its dehydration and deep protection.

Gas chromatograph (60 m DBWax-60N, 50-250° C. 4° C./min):

t_{R}=40.50 (rac-36-Z), 40.55 (rac-37-Z), 40.85 (rac-37-E), 40.95 min (rac-36-E).

The E/Z-ratio was determined by integration of the characteristic ¹H-NMR-signal for the side chain-vinyl proton at 5.08 (E-isomer) and 4.835 ppm (Z-isomer) determined to be 55:45.

NMR-Analysis showed

(2SR,3RS)-3-methyl-4-[(E,1RS,2SR,6RS,7RS)tricyclo[5.2.1.0^{2,6}]dec-3-en-8-ylidene]butan-2-ol (rac-36-E) and (2RS,3SR)-3-methyl-4-[(Z,1RS,2SR,6RS,7RS)tricyclo[5.2.1.0^{2,6}]dec-3-en-8-ylidene]butan-2-ol (rac-37-Z) as main isomer and rac-37-E and rac-36-Z as side isomer:

¹H-NMR (300 MHz, CDCl₃):

δ=3.37-3.53 (m, 1H), 5.47-5.53 (m, 1H), 5.65-5.71 ppm (m, 1H).

rac-36-E assigned: δ=0.925 (d, J=7 Hz), 1.15 (d, J=6 Hz), 2.35 (br. s), 5.08 ppm (dt, J=10, 2.25 Hz).

rac-37-Z assigned: δ=0.96 (d, J=7 Hz), 1.17 (d, J=6 Hz), 2.68 (br. s), 4.835 ppm (d, J=10 Hz).

¹³C-NMR (75 MHz, CDCl₃):

rac-36-E assigned: δ=16.8 (q), 20.0 (q), 32.45 (t), 35.75 (t), 39.05 (t), 40.9 (d), 41.7 (d), 43.8 (d), 51.8 (d), 55.2 (d), 71.7 (d), 119.7 (d), 132.25 (d), 132.35 (d), 148.25 ppm (s).

rac-37-Z assigned: δ=17.4 (q), 19.9 (q), 32.35 (t), 38.35 (t), 39.2 (t), 40.6 (d), 42.45 (d), 43.3 (d), 47.05 (d), 55.1 (d), 71.55 (d), 120.5 (d), 132.15 (d), 132.4 (d), 147.75 ppm (s).

The side isomer rac-37-E and rac-36-Z appear with signals of lower intensity.

MS:

rac-36-E: m/z (%)=218 (4, M⁺), 174 (58), 173 (82), 131 (20), 117 (22), 107 (100), 105 (28), 91 (55), 79 (53), 77 (27), 67 (56).

rac-37-Z: m/z (%)=218 (2, M+), 174 (72), 173 (98), 131 (27), 117 (35), 107 (100), 105 (35), 91 (66), 79 (74), 77 (34), 67 (77).

Alcohol Mixture rac-38-E/rac-39-Z/rac-39-E/rac-38-Z (Protocol 7)

Production was analogously to the above production of the alcohol mixture rac-C/rac-33-Z/rac-D/rac-32-Z from alcohol mixture rac-36-E/rac-37-Z/rac-37-E/rac-36-Z.

Gas chromatograph (60 m DBWax-60N, 60-250° C. 4° C./min):

t_{R}=40.85 (rac-38-Z), 41.00 (rac-39-Z), 41.45 (rac-39-E), 41.475 min (rac-38-E).

The E/Z-ratio was determined by integration of the characteristic ¹H-NMR-signal for the side chain-vinyl proton at 5.085 (E-isomer) and 4.85 ppm (Z-isomer) and was determined to be 55:45.

NMR-Analysis showed

(2RS,3RS)-3-methyl-4-[(E,1RS,2SR,6RS,7RS)tricyclo[5.2.1.0^{2,6}]dec-3-en-8-ylidene]butan-2-ol (rac-38-E) and (2SR,3SR)-3-methyl-4-[(Z,1RS,2SR,6RS,7RS)tricyclo[5.2.1.0^{2,6}]dec-3-en-8-ylidene]butan-2-ol (rac-39-Z) as main isomer and rac-39-E and rac-38-Z as side isomers:

¹H-NMR (300 MHz, CDCl₃):

δ=3.54-3.64 (m, 1H), 5.48-5.54 (m, 1H), 5.65-5.71 ppm (m, 1H). rac-38-E assigned: δ=0.965 (d, J=7 Hz), 1.12 (d, J=6 Hz), 2.34 (br. s), 5.085 ppm (ddd, J=10, 5,2 Hz).

rac-39-Z assigned: δ=1.00 (d, J=7 Hz), 1.115 (d, J=6.5 Hz), 2.67 (br. s), 4.85 ppm (d, J=9.5 Hz).

¹³C-NMR (75 MHz, CDCl₃):

rac-38-E assigned: δ=16.6 (q), 20.15 (q), 32.3 (t), 35.75 (t), 39.1 (t), 40.8 (d), 40.95 (d), 43.85 (d), 51.8 (d), 55.2 (d), 72.05 (d), 119.3 (d), 132.25 (d), 132.4 (d), 146.85 ppm (s).

rac-39-Z assigned: δ=17.3 (q), 19.95 (q), 32.25 (t), 38.4 (t), 39.2 (t), 40.65 (d), 41.25 (d), 43.35 (d), 47.0 (d), 55.1 (d), 72.0 (d), 119.75 (d), 132.2 (d), 132.45 (d), 146.45 ppm (s).

The side isomers rac-39-E and rac-38-Z were displayed by signals of lower intensity.

MS:

rac-38-E: m/z (%)=218 (2, M⁺), 174 (58), 173 (83), 131 (24), 117 (26), 107 (100), 105 (27), 91 (56), 79 (56), 77 (25), 67 (67).

rac-39-Z: m/z (%)=218 (2, M+), 174 (54), 173 (86), 131 (24), 117 (25), 107 (100), 105 (28), 91 (62), 79 (57), 77 (27), 67 (69).

Alcohol Mixture A/30-Z (Protocol 8)

The production occurred analogously to the preceding production of the alcohol mixture rac-A/rac-31-Z/rac-B/rac-30-Z, that is, from ketone 13 and bromide 23 via coupling alcohol 28 and the protection thereof by dehydration.

Gas chromatograph (60 m HP5, 60-250° C. 4° C./min):

t_{R}=30.80 (19% 30-Z), 31.05 min (81% A).

(2S,3R)-3-methyl-4-[(E,1R,2R,6R,7R)tricyclo[5.2.1.0^{2,6}]dec-4-en-8-ylidene]butan-2-ol (A=30-(2S,3R)-3-methyl-4-[(Z,1R,2R,6R,7R)tricyclo[5.2.1.0^{2,6}]dec-4-en-8-ylidene]butan-2-ol (30-Z):

¹H-NMR (300 MHz, CDCl₃):

δ=3.41-3.55 (m, 1H), 5.44-5.50 (m, 1H), 5.64-5.70 ppm (m, 1H).

A (=30-E): δ=0.925 (d, J=7 Hz), 1.145 (d, J=6 Hz), 2.41 (br. s, 0.8H), 5.08 ppm (d″d″, J=10,2.5 Hz, 0.8H).

30-Z: δ=0.94 (d, J=7 Hz), 1.17 (d, J=6 Hz), 4.85 ppm (d, J=10 Hz, 0.2H).

¹³C-NMR (75 MHz, CDCl₃):

A (=30-E): δ=16.8 (q), 20.0 (q), 32.4 (t), 35.7 (t), 39.7 (t), 41.65 (d), 43.15 (d), 43.2 (d), 49.3 (d), 55.8 (d), 71.7 (d), 119.6 (d), 131.4 (d), 132.25 (d), 148.45 ppm (s).

30-Z: δ=17.3 (q), 19.95 (q), 32.3 (t), 38.15 (t), 39.75 (t), 42.35 (d), 42.95 (d), 43.15 (d), 44.35 (d), 55.2 (d), 71.55 (d), 120.4 (d), 131.4 (d), 132.35 (d), 147.5 ppm (s).

MS:

A (=30-E): m/z (%)=218 (8, M⁺), 174 (17), 173 (53), 151 (18), 108 (14), 107 (100), 91 (22), 79 (24), 67 (55).

30-Z: m/z (%)=218 (8, M⁺), 174 (15), 173 (49), 151 (16), 108 (13), 107 (100), 91 (22), 79 (23), 67 (57).

Alcohol Mixture C/32-Z (Protocol 8)

The production occurred analogously to the preceding production of alcohol mixture rac-C/rac-33-Z/rac-D/rac-32-Z from alcohol mixture A/30-Z

Gas chromatograph (60 m HP5, 60-250° C. 4° C./min):

t_{R}=31.35 (20% 32-Z), 31.55 min (80% C).

(2R,3R)-3-methyl-4-[(E,1R,2R,6R,7R)tricyclo[5.2.1.0^{2,6}]dec-4-en-8-ylidene]butan-2-ol (C=32-E), (2R,3R)-3-methyl-4-[(Z, 1 R,2R,6R,7R)tricyclo[5.2.1.0^{2,6}]dec-4-en-8-ylidene]butan-2-ol (32-Z):

¹H-NMR (300 MHz, CDCl₃): δ=3.54-3.66 (m, 1H), 5.44-5.50 (m, 1H), 5.65-5.71 ppm (m, 1H).

C (=32-E): δ=0.965 (d, J=7 Hz), 1.12 (d, J=6 Hz), 2.39 (br. s, 0.8H), 5.09 ppm (dt, J=10, 2 Hz, 0.8H)

32-Z: δ=0.985 (d, J=7 Hz), 1.16 (d, J=6 Hz), 4.86 ppm (d, J=9.5 Hz, 0.2H) ¹³C-NMR (75 MHz, CDCl₃):

C (=32-E):δ=16.65 (q), 20.15 (q), 32.25 (t), 35.7 (t), 39.7 (t), 40.8 (d), 43.15 (d), 43.25 (d), 49.3 (d), 55.85 (d), 72.05 (d), 119.15 (d), 131.5 (d), 132.25 (d), 147.1 ppm (s).

32-Z: δ=17.3 (q), 20.55 (q), 32.3 (t), 38.15 (t), 39.7 (t), 41.55 (d), 42.95 (d), 43.15 (d), 44.3 (d), 55.0 (d), 72.0 (d), 119.9 (d), 131.35 (d), 132.45 (d), 146.2 ppm (s).

MS:

C (=32-E): m/z (%)=218 (8, M⁺), 174 (17), 173 (52), 151 (14), 108 (14), 107 (100), 91 (26), 79 (30), 67 (67).

32-Z: m/z (%)=218 (5, M+), 174 (15), 173 (47), 151 (12), 108 (12), 107 (100), 91 (25), 79 (30), 67 (72).

Alcohol Mixture B/31-Z (Protocol 9)

Production occurred analogously to the preceding production of the alcohol mixture rac-A/rac-31-Z/rac-B/rac-30-Z, that is, from ketone 13 and bromide ent-23 via coupling alcohol 29 and the dehydration and the protection thereof.

Gas chromatograph (60 m HP5, 60-250° C. 4° C./min):

t_{R}=30.95 (70% 31-Z), 31.00 min (30% B).

(2R,3S)-3-methyl-4-[(E,1 R,2R,6R,7R)tricyclo[5.2.1.0^{2,6}]dec-4-en-8-ylidene]butan-2-ol (B=31-E), (2R,3S)-3-methyl-4-[(Z, 1R,2R,6R,7R)tricyclo[5.2.1.0^{2,6}]dec-4-en-8-ylidene]butan-2-ol (31-Z):

¹H-NMR (300 MHz, CDCl₃):

δ=3.38-3.53 (m, 1H), 5.44-5.50 (m, 1H), 5.65-5.71 ppm (m, 1H).

B (=31-E): δ=0.925 (d, J=7 Hz, 0.9H), 1.155 (d, J=6 Hz), 2.42 (br. s), 5.075 ppm (dt, J=10, 2 Hz, 0.3H).

31-Z: δ=0.97 (d, J=7 Hz, 2.1H), 1.17 (d, J=6 Hz), 2.34 (ddq (10 Linien), J=7,7, 10 Hz, 0.7H), 2.73 (br. s), 4.85 ppm (d, J=10 Hz, 0.7H). ¹³C-NMR (75 MHz, CDCl₃):

B (=31-E): δ=16.7 (q), 19.95 (q), 31.95 (t), 35.45 (t), 39.65 (t), 41.45 (d), 43.1 (d), 43.15 (d), 49.5 (d), 56.4 (d), 71.65 (d), 119.35 (d), 131.35 (d), 132.3 (d), 148.65 ppm (s).

31-Z: δ=17.4 (q), 19.95 (q), 32.35 (t), 38.3 (t), 39.7 (t), 42.25 (d), 42.9 (d), 43.05 (d), 44.55 (d), 55.25 (d), 71.5 (d), 120.25 (d), 131.25 (d), 132.45 (d), 147.85 ppm (s).

MS:

B (=31-E): m/z (%)=218 (7, M+), 174 (12), 173 (40), 151 (18), 108 (13), 107 (100), 91 (21), 79 (24), 67 (51).

31-Z: m/z (%)=218 (8, M+), 174 (19), 173 (58), 151 (12), 108 (14), 107 (100), 91 (25), 79 (28), 67 (61).

Alcohol Mixture D/33-Z (Protocol 9)

Production occurred analogously to the preceding production of the alcohol mixture rac-C/rac-33-Z/rac-D/rac-32-Z from alcohol mixture B/31-Z

Gas chromatograph (60 m HP5, 60-250° C. 4° C./min):

t_{R}=31.40 (70% 33-Z), 31.50 min (30% D).

(2S,3S)-3-methyl-4-[(E,1 R,2R,6R,7R)tricyclo[5.2.1.0^{2,6}]dec-4-en-8-ylidene]butan-2-ol (D=33-E), (2S,3S)-3-methyl-4-[(Z, 1 R,2R,6R,7R)tricyclo[5.2.1.0^{2,6}]dec-4-en-8-ylidene]butan-2-ol (33-Z):

¹H-NMR (300 MHz, CDCl₃):

δ=3.54-3.64 (m, 1H), 5.44-5.50 (m, 1H), 5.66-5.72 ppm (m, 1H).

D (=33-E): δ=0.965 (d, J=7 Hz, 0.9H), 1.14 (d, J=6.5 Hz), 2.40 (br. s), 5.07 ppm (dt, J=10, 2.25 Hz, 0.3H.

33-Z: δ=1.005 (d, J=7 Hz, 2.1H), 1.12 (d, J=6.5 Hz), 2.50 (ddq (10 Linien), J=6.5, 6.5, 10 Hz), 2.73 (br. s), 4.86 ppm (d, J=10 Hz, 0.7H).

¹³C-NMR (75 MHz, CDCl₃):

D (=33-E): δ=16.65 (q), 20.35 (q), 32.0 (t), 35.55 (t), 39.7 (t), 40.8 (d), 43.15 (d), 43.2 (d), 49.45 (d), 56.05 (d), 72.25 (d), 119.2 (d), 131.4 (d), 132.35 (d), 147.25 ppm (s).

33-Z: δ=17.35 (q), 19.95 (q), 32.25 (t), 38.3 (t), 39.7 (t), 41.2 (d), 42.95 (d), 43.05 (d), 44.55 (d), 55.3 (d), 72.0 (d), 119.5 (d), 131.3 (d), 132.45 (d), 146.75 ppm (s).

MS:

D (=33-E): m/z (%)=218 (6, M⁺), 174 (12), 173 (43), 151 (14), 108 (13), 107 (100), 91 (24), 79 (28), 67 (68).

33-Z: m/z (%)=218 (7, M⁺), 174 (18), 173 (56), 151 (11), 108 (14), 107 (100), 91 (24), 79 (31), 67 (75).

Alcohol Mixture ent-A/ent-30-Z (Protocol 10)

Production occurred analogously to the preceding production of the alcohol mixture rac-A/rac-31-Z/rac-B/rac-30-Z, that is from ketone ent-13 and bromide ent-23 via coupling alcohol ent-28 and the dehydration thereof and the protection thereof.

Gas chromatograph (60 m HP5, 60-250° C. 4° C./min):

t_{R}=30.80 (20% ent-30-Z), 31.05 min (80% ent-A).

(2R,3S)-3-methyl-4-[(E,1S,2S,6S,7S)tricyclo[5.2.1.0^{2,6}]dec-4-en-8-ylidene]butan-2-ol (ent-A), (2R,3S)-3-methyl-4-[(Z, 1 S,2S,6S,7S)tricyclo[5.2.1.0^{2,6}]dec-4-en-8-ylidene]butan-2-ol (ent-30

The spectral data correspond to those of alcohol mixture A/30-Z.

Alcohol Mixture ent-C/ent-32-Z (Protocol 10)

Production occurred analogously to the preceding production of the alcohol mixture rac-C/rac-33-Z/rac-D/rac-32-Z from the alcohol mixture ent-A/ent-30-Z

Gas chromatograph (60 m HP5, 60-250° C. 4° C./min):

t_{R}=31.35 (20% ent-32-Z), 31.55 min (80% ent-C).

(2S,3S)-3-methyl-4-[(E,1S,2S,6S,7S)tricyclo[5.2.1.0^{2,6}]dec-4-en-8-ylidene]butan-2-ol (ent-C), (2S,3S)-3-methyl-4-[(Z, 1 S,2S,6S,7S)tricyclo[5.2.1.0^{2,6}]dec-4-en-8-ylidene]butan-2-ol (ent-32-Z):

The spectral data corresponds to that of alcohol mixture C/32-Z.

Alcohol Mixture ent-B/ent-31-Z (Protocol 11)

Production occurred analogously to the preceding production of the alcohol mixture rac-A/rac-31-Z/rac-B/rac-30-Z, that is, from ketone ent-13 and bromide 23 via coupling alcohol ent-29 and the dehydration and the protection thereof.

Gas chromatograph (60 m HP5, 60-250° C. 4° C./min):

t_{R}=30,95 (71% ent-31-Z), 31,00 (29% ent-B).

(2S,3R)-3-methyl-4-[(E,1S,2S,6S,7S)tricyclo[5.2.1.0^{2,6}]dec-4-en-8-ylidene]butan-2-ol (ent-B), (2S,3R)-3-methyl-4-[(Z,1S,2S,6S,7S)tricyclo[5.2.1.0^{2,6}]dec-4-en-8-ylidene]butan-2-ol (ent-31-Z):

The spectral data correspond to those of alcohol mixture B/31-Z.

Alcohol Mixture ent-D/ent-33-Z (Protocol 11)

Production occurred analogously to the preceding production of the alcohol mixture rac-C/rac-33-Z/rac-D/rac-32-Z from the alcohol mixture ent-B/ent-31-Z

Gas chromatograph (60 m HP5, 60-250° C. 4° C./min):

t_{R}=31.40 (71% ent-33-Z), 31.50 (29% ent-D).

(2R,3R)-3-methyl-4-[(E,1S,2S,6S,7S)tricyclo[5.2.1.0^{2,6}]dec-4-en-8-ylidene]butan-2-ol (ent-D),

(2R,3R)-3-methyl-4-[(Z,1 S,2S,6S,7S)tricyclo[5.2.1.0^{2,6}]dec-4-en-8-ylidene]butan-2-ol (ent-33

The spectral data correspond to those of alcohol mixture D/33-Z.

### Example 2

### Sensorial Evaluation of Inventive Isomers

2.1 (2S,3R)-3-Methyl-4-[(E,1R,2R,6R,7R)tricyclo[5.2.1.0^{2,6}]dec4-en-8-ylidene]butan-2-ol

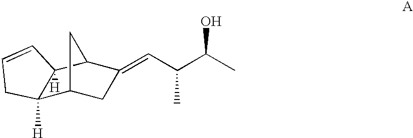

Fragrance: very strong sandal, animal

Sensory (smell) threshold value: 5 μg/l water

2.2 (2R,3S)-3-Methyl-4-[(E,1S,2S,6S,7S)tricyclo[5.2.1.0^{2,6}]dec-4-en-8-ylidene]butan-2-ol (ent-A)

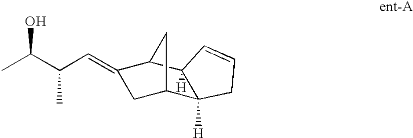

Fragrance: weak woody

Sensory (smell) threshold value: 168 μg/l water

2.3 (2R,3S)-3-Methyl-4-[(E,1R,2R,6R,7R)tricyclo[5.2.1.0^{2,6}]dec-4-en-8-ylidene]butan-2-ol (B)

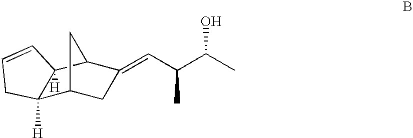

Fragrance: sandal, animal

Sensory (smell) threshold value: 70 μg/l water

2.4 (2S,3R)-3-Methyl-4-[(E,1S,2S,6S,7S)tricyclo[5.2.1.0^{2,6}]dec-4-en-8-ylidene]butan-2-ol (ent-B)

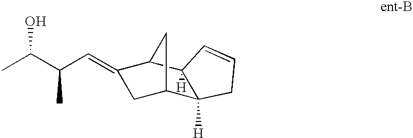

Fragrance: woody

Sensory (smell) threshold value: 72 μg/l water

2.5 (2R,3R)-3-Methyl-4-[(E,1 R,2R,6R,7R)tricyclo[5.2.1.0^{2,6}]dec-4-en-8-ylidene]butan-2-ol (C)

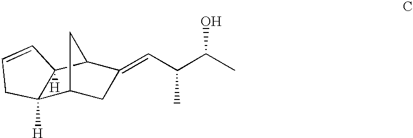

Fragrance: sandal, floral

Sensory (smell) threshold value: 137 μg/l water

2.6 (2S,3S)-3-Methyl-4-[(E,1S,2S,6S,7S)tricyclo[5.2.1.0^{2,6}]dec-4-en-8-ylidene]butan-2-ol (ent-C)

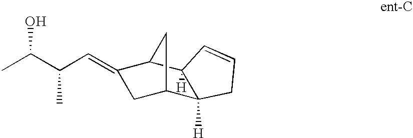

Fragrance: very weak floral

Sensory (smell) threshold value: 159 μg/l water

2.7 (2S,3S)-3-Methyl-4-[(E,1R,2R,6R,7R)tricyclo[5.2.1.0^{2,6}]dec-4-en-8-ylidene]butan-2-ol (D)

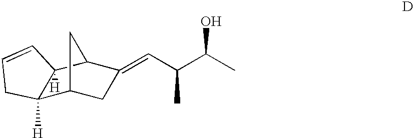

Fragrance: strong sandal, floral

Sensory (smell) threshold value: 46 μg/l water

2.8 (2R,3R)-3-Methyl-4-[(E,1S,2S,6S,7S)tricyclo[5.2.1.0^{2,6}]dec-4-en-8-ylidene]butan-2-ol (ent-D)

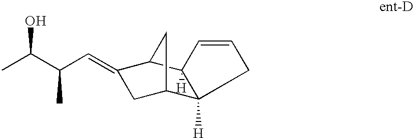

Fragrance: weak floral

Sensory (smell) threshold value: 204 μg/l water

What is claimed is:

1. Compound of the Formula
(2R/S, 3R/S)-3-Methyl-4-[(E,1R/S,2R/S,6R/S,7R/S)tricyclo[5.2.1.0^{2,6}]dec-4-en-8-ylidene]butan-2-ol,
wherein respectively independently from each other the following applies:
(2R/S, 3R/S) means (2R,3R), (2R,3S), (2S,3R) or (2S,3S) and
(E,1R/S,2R/S,6R/S,7R/S) means (E,1R,2R,6R,7R) or (E,1S,2S,6S,7S).

2. Compound according to claim 1 with the configuration (E,1R,2R,6R,7R).

3. Compound according to claim 2 with the configuration (2S, 3R) or (2S,3S).

4. Compound according to claim 1 with the configuration (E,1S,2S,6S,7S).

5. Mixture of multiple configurational isomers of a compound according to one of the preceding claims.

6. Mixture, including
a configurational isomer or multiple configurational isomers of a compound according to one of claims 1-4 as well as
one or more compounds of the formula
3-methyl-4-[(E,1R/S,2S/R,6R/S,7R/S)tricyclo[5.2.1.0^{2,6}]dec-3-en-8-ylidene]-butan-2-ol
or the Formula
3-methyl-4-[(Z,1R/S,2R/S,6R/S,7R/S)tricyclo[5.2.1.0^{2,6}]dec-4-en-8-ylidene]-butan-2-ol
or the Formula
3-methyl-4-[(Z,1R/S,2S/R,6R/S,7R/S)tricyclo[5.2.1.0^{2,6}]dec-3-en-8-ylidene]-butan-2-ol
wherein the proportions are so selected, that the sensorial characteristics of the mixture are primarily determined by the configurational isomer(s) of the compound according to one of claims 1-4.

7. Organoliptic composition including a sensorially effective amount of the compound according to one of claims 1-4 or a mixture according to one of claims 5-6.

8. Use of a compound according to one of claims 1-4 or a mixture according to one of claims 5-6 as fragrance or flavor substance.

9. Process for modification of the sensorial characteristics of a fragrance or flavor composition, wherein one or more components of the fragrance or flavor composition are mixed with a sensorially effective amount of the compound according to one of claims 1-4 or a mixture according to one of claims 5-6.
